(19) United States
(12) Patent Application Publication
(10) Pub. No.: US 2023/0340609 A1
(43) Pub. Date: Oct. 26, 2023

(54) CANCER DETECTION, MONITORING, AND REPORTING FROM SEQUENCING CELL-FREE DNA

(71) Applicant: Claret Bioscience, LLC, Santa Cruz, CA (US)

(72) Inventors: Kelly M. HARKINS KINCAID, Santa Cruz, CA (US); Charles VASKE, Santa Cruz, CA (US)

(21) Appl. No.: 18/016,957

(22) PCT Filed: Jul. 20, 2021
(86) PCT No.: PCT/US2021/042360

§ 371 Date: Jan. 19, 2023

Related U.S. Application Data
(60) Provisional application No. 63/054,671, filed on Jul. 21, 2020.

(51) Int. Cl. C12Q 1/6886

(52) U.S. Cl. CPC C12Q 1/6886; C12Q 2600/156; C12Q 2600/158; C12Q 2600/154

(57) ABSTRACT

Provided in part are techniques for cancer detection, monitoring, and reporting from sequencing cell-free DNA in plasma samples. Such detection can be informed from patient-specific circulating tumor cell (CTC) somatic genomic, epigenetic, and/or transcriptomic modifications. These techniques can be used to aid treatment decision support, diagnosis, and/or prognosis of cancer.

### RELATED PATENT APPLICATION(S)

This patent application is a 35 U.S.C. 371 national phase application of International Patent Cooperation Treaty (PCT) Application No. PCT/US2021/042360, filed on Jul. 20, 2021, entitled CANCER DETECTION, MONITORING, AND REPORTING FROM SEQUENCING CELL-FREE DNA, naming Kelly M. Harkins Kincaid et al. as inventors, and designated by attorney docket no. CBS-2005PCT, which claims the benefit of U.S. provisional patent application No. 63/054,671 filed on Jul. 21, 2020, entitled CANCER DETECTION, MONITORING, AND REPORTING FROM SEQUENCING CELL-FREE DNA, naming Kelly M. HARKINS KINCAID et al. as inventors, and designated by attorney docket no. CBS-2005-PV. The entire content of the foregoing patent application is incorporated herein by reference for all purposes, including all text, tables and drawings.

### FIELD

The technology relates in part to techniques for cancer detection, monitoring, and reporting from sequencing cell-free DNA in plasma samples. Such detection can be informed from patient-specific circulating tumor cell (CTC) somatic genomic, epigenetic, and/or transcriptomic modifications. These techniques can be used to aid treatment decision support, diagnosis, and/or prognosis of cancer.

### BACKGROUND

Cancer harbors a multitude of genomic, epigenetic, and transcriptomic modifications compared to germline data. Detection and monitoring of cancer via tumor biopsies can be invasive, dangerous and expensive. Imaging data can be uninformative and expensive. And in the case of unknown location of the cancer, imaging and biopsy can be impossible.

However, blood plasma is easily accessible with minimum risk to patients, and the cell-free DNA (cfDNA) in blood plasma contains circulating tumor DNA (ctDNA) as a fraction of the DNA molecules. The molecules that are present in a cfDNA sample are heavily influenced by the cellular state of the originating cells, including but not limited to protein binding, chromatin structure, active nucleases, and other factors. Therefore, the particular ends of a DNA fragment and its genomic location can reveal information about the originating cell.

Circulating tumor cells (CTC) can also be isolated from a patient's blood, and the genomic, epigenetic, transcriptomic, and proteomic deviations from normal body cells can be ascertained using molecular biology and DNA/RNA sequencing techniques.

### SUMMARY

Provided in certain aspects are methods comprising obtaining a sample from a subject, the sample comprising DNA comprising circulating tumor DNA (ctDNA) and cell-free DNA (cfDNA); sequencing the DNA to generate sequencing reads; detecting at least one ctDNA property of (i) a patient-specific ctDNA property and (ii) a general ctDNA property; and determining at least some of the sequencing reads as ctDNA sequencing reads based on the at least one ctDNA property.

Also provided in certain aspects are methods comprising obtaining a sample from a subject, the sample comprising DNA comprising circulating tumor DNA (ctDNA) and cell-free DNA (cfDNA); sequencing the DNA to generate sequencing reads; detecting (i) one or more ctDNA properties of a patient-specific ctDNA property and/or (ii) one or more ctDNA properties of a general ctDNA property; and determining at least some of the sequencing reads as ctDNA sequencing reads based on the one or more ctDNA properties.

Also provided in certain aspects are methods comprising a) obtaining a first sample from a subject, the first sample comprising DNA comprising circulating tumor DNA (ctDNA); b) obtaining a second sample from a subject, the second sample comprising DNA comprising circulating tumor DNA (ctDNA) and cell-free DNA (cfDNA); c) sequencing the DNA obtained in (a) and (b) to generate sequencing reads; d) detecting at least one ctDNA property of (i) a patient-specific ctDNA property and (ii) a general ctDNA property; and e) determining at least some of the sequencing reads as ctDNA sequencing reads based on the at least one ctDNA property.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows a representative trace of single-cell single-stranded prep (“SRSLY”; CLARETBIO, Santa Cruz, CA) libraries. (L) 1 Kb Ladder (1) 10 cell input by bulk dilution (2) 1 cell input by bulk dilution (3) 1 cell input from EPIC sorted cell culture (C) No template control. In brief cell(s) were incubated with Triton-X and diluted DNase I for 5 minutes at 37° C., followed by SDS and proteinase K at 55° C. for 45 minutes. After DNA purification, SRSLY next generation sequencing (NGS) library prep protocol was performed on all samples. DNA mass centered around 350 bp represents the sequencing libraries. NGS adapter dimers are present at around 150 bp.

### DETAILED DESCRIPTION

Circulating tumor DNA (ctDNA) can be distinguished and classified from cell-free DNA (cfDNA) in a sequenced plasma blood sample, for example using statistical, machine learning, and/or artificial intelligence techniques. Certainty of DNA origin can be assigned to each sequenced molecule, and/or to the sample as a whole, using features derived from a patient's own particular CTCs, general characteristics of ctDNA versus cfDNA not specific to a patient, or a combination of patient-specific and general properties of ctDNA.

Patient-Specific ctDNA Properties

One or more patient-specific ctDNA properties can be derived from a sampling of one or more CTCs (e.g., a single CTC) subjected to investigation.

An example of a ctDNA property is tumor somatic mutation. Tumor somatic mutations not present in a patient's germline can be directly detectable in cfDNA sequencing data, with or without error-correcting library preparations.

Another example of a ctDNA property is genomic DNA accessibility at a particular site not typically found in blood or other subset of tissues. DNA accessibility is influenced by protein binding, e.g. nucleosomes or transcription factors, and other DNA binding proteins and DNA superstructure. This property can be assessed by DNase treatment, ATAC-seq, or other means, and compared to a patient's normal tissues (e.g., peripheral blood mononuclear cell, skin punch, etc.) or by comparison to databases of normal tissues. DNA accessibility can be detectable by the coverage amount and/or end status of reads that cover the particular loci. DNA accessibility can be assessed in terms of A/B compartments of open and closed chromatin. A/B compartment status can be assessed with techniques such as Hi-C, Dip-C, ATAC-seq, methylation status, DNase hypersensitivity sequencing, and others. A/B compartment status can be assessed over the entire genome, or at specific regions, such as compartment boundaries or transitions, promoter or other regulatory sites, or genes or other loci of interest.

Another example of a ctDNA property is genomic DNA accessibility at a group of sites that is differential from an expectation set by patient data or databases of other samples. Examples include but are not limited to groups of nucleosome positions, groups of transcription factor binding sites, loci clustered together in the 3D organization of the cell nucleus, and others. This property can be detectable by combining data for multiple sites through probabilistic, statistical, machine learning, or AI methods.

Another example of a ctDNA property is methylation of genomic DNA. Methylation status can be detectable by certain sequencing methods, including but not limited to bisulfite treatment, enzymatic methylation sequencing, nanopore sequencing, and others. In some embodiments, a methylation analysis may include methylation sequencing (Methyl-Seq). Methylation sequencing typically includes a treatment to deaminate cytosine in sample nucleic acid. Deamination refers to the removal of an amino group from a molecule. Such treatment produces two different results based on the methylation status of cytosine 1) unmethylated cytosine residues are converted to uracil and 2) methylated cytosine (5′ methylcytosine, 5-mC, 5-hmC) residues remain unmodified by the treatment. In some assays, deamination treatment may be followed by nucleic acid amplification (e.g., PCR) and/or nucleic acid sequencing (e.g., massively parallel sequencing) to reveal the methylation status of cytosine residues in a gene-specific analysis or whole genome analysis. Unmethylated cytosine residues converted to uracil typically are amplified in a subsequent amplification reaction as thymine residues, whereas the methylated cytosine residues are amplified as cytosine residues. Comparison of sequence information between a reference genome and deamination treated nucleic acid can provide information about cytosine methylation patterns.

Deamination treatment may comprise a chemical-based treatment and/or an enzyme-based treatment. Chemical based treatments may include sodium bisulfite treatments, also referred to as bisulfite conversion (e.g., ZYMO's EZ METHYLATION-LIGHTNING Kit). Enzyme-based treatments may include use of a deaminase enzyme (e.g., a cytidine deaminase; NEBNext® Enzymatic Methyl-seq (EM-seq™) (NEB #E7120)). Deaminase enzymes may include APOBEC (apolipoprotein B mRNA editing enzyme, catalytic polypeptide-like) which is a family of cytidine deaminases. Bisulfite treatments are generally considered harsh, often resulting in denaturing, shearing, and/or loss of the sample nucleic acid, while enzyme-based treatments are considered as mild relative to bisulfite treatment and can minimize damage to sample nucleic acid. Without being limited by theory, bisulfite treatment may be suitable for sample nucleic acid comprising short nucleic acid fragments (e.g., fragments less than about 250 bp) where the treatment results in minimal shearing and/or loss, in certain instances.

Another example of a ctDNA property is a transcriptome-wide profile of the CTCs, for example through RNA sequencing. Such transcriptome-wide profiles can reveal the activity of transcription factors, and provide data about nucleosome positioning, chromatin structure, and the 3D organization of the nucleus. This profile can be determined by the genomic location of read ends and coverage depth that indicate the protein binding status (e.g., nucleosome) of a fragment and of genomic loci in the tumor versus those in the rest of the body.

Another example of a ctDNA property is copy number variations (CNVs) and/or specific gene expression levels (e.g., RNA levels or specific protein levels), for example expression levels of nucleases, apoptosis pathway members, necrosis pathway members, and members of other cellular pathways implicated in cell death and DNA degradation or DNA protection. The base composition and genomic DNA context, of native, unmodified ends of DNA fragments can be influenced by the nucleases and other cellular pathways that release DNA from a cell. Read depth coverage and read end locations can indicate the epigenetic state that leads to the RNA and protein expression states that are measured via CTC profiling.

Another example of a ctDNA property is epigenetic protein modification such as histone methylation, acetylation, and phosphorylation that indicate chromatin differences between a patient's tumor cell and normal tissues can be assayed with immunoprecipitation or other means. The epigenetic state of the originating chromosomal DNA can influence cfDNA fragment ends, lengths, and locations of genomic coverage.

Properties of ctDNA can be determined by synthetic fragmentomic methods. CTCs can be digested, for example with a patient's own serum, fluid from a tumor, or a panel of selected enzymes such as nucleases. The resulting fragmented nucleic acids can then be analyzed and informative features such as those discussed herein, including epigenetic and fragmentomic features (e.g., nucleosome positioning, transcription factor-bound DNA) can be determined.

General Properties of ctDNA Versus cfDNA

General properties of ctDNA versus cfDNA derived from databases of non-patient-specific information can include one or more properties assessed for a specific patient, or all of the properties that could be assessed for a specific patient, as well as other properties including but not limited to fragment length (including fragment length ratios) and fragment ends (e.g., fragment overhang sequence, fragment overhang length, fragment overhang length ratios, fragment overhang directionality, genomic location of fragment ends). These properties can be associated with or correlated with features of the tumor, including but not limited to tissue of origin of the tumor, tumor stage, tumor type, and other features. Ratios can be ratios of “short” to “long” fragments or overhangs, where the threshold for “short” and “long” can be selected appropriately.

Properties of ctDNA can be determined by synthetic fragmentomic methods. As discussed above, CTCs can be digested, for example with serum (including a patient's own serum), fluid from a tumor, or a panel of selected enzymes such as nucleases. The resulting fragmented nucleic acids can then be analyzed and informative features such as those discussed herein, including epigenetic and fragmentomic features (e.g., nucleosome positioning, transcription factor-bound DNA) can be determined. These ctDNA properties can be analyzed for a plurality of samples, and general correlations, trends, and other properties can be determined.

Sample Analysis

A patient sample of cfDNA can be assessed in many ways, including but not limited to off-the-shelf DNA extraction and library preparation, single stranded DNA (ssDNA) library preparation, methyl-Seq extraction and library preparation, duplex, or other error-correcting sequencing methods, native, unmodified end preservation library preparation, detection of double stranded DNA (dsDNA) overhangs, or of single-stranded state.

Analysis can include methods described in PCT Patent Publication Nos. WO 2019/140201 and WO 2020/206143, each of which is incorporated herein by reference.

Data Analysis

Given one or more DNA properties, such as those patient-specific or general properties discussed herein, and a sequenced sample of a patient's cfDNA, ctDNA can be distinguished from cfDNA by approaches including but not limited to assessing likelihood of origin of individual molecules, assessing subsets of the cfDNA fragments for ctDNA origin, and/or assessing the entire set of cfDNA reads for likelihood of tumor origin.

Analyses can be combined in a longitudinal, time-series, manner with repeated plasma cfDNA samples over multiple time points.

Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single or double stranded. Single stranded DNA (ssDNA), for example, can be generated by denaturing double stranded DNA by heating or by treatment with alkali, for example. Accordingly, in some embodiments, ssDNA is derived from double-stranded DNA (dsDNA). In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

Nucleic acid (e.g., nucleic acid targets, single-stranded nucleic acid, oligonucleotides, overhangs, and the like) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). In some embodiments, one or more thymine (T) bases are replaced by uracil (U) in a sequencing and/or library adapter, or a component thereof, and is/are complementary to adenine (A). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions. In certain instances, a nucleotide sequence may be partially complementary to a target, in which not all nucleotides are complementary to every nucleotide in the target nucleic acid in all the corresponding positions.

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=#of identical positions/total #of positions×100). When a position in one sequence is occupied by the same nucleotide as the corresponding position in the other sequence, then the molecules are identical at that position.

In some embodiments, nucleic acids in a mixture of nucleic acids are analyzed. A mixture of nucleic acids can comprise two or more nucleic acid species having the same or different nucleotide sequences, different lengths, different origins (e.g., genomic origins, fetal vs. maternal origins, cell or tissue origins, cancer vs. non-cancer origin, tumor vs. non-tumor origin, host vs. pathogen, host vs. transplant, host vs. microbiome, sample origins, subject origins, and the like), different overhang lengths, different overhang types (e.g., 5′ overhangs, 3′ overhangs, no overhangs), or combinations thereof. In some embodiments, a mixture of nucleic acids comprises single-stranded nucleic acid and double-stranded nucleic acid. In some embodiment, a mixture of nucleic acids comprises DNA and RNA. In some embodiment, a mixture of nucleic acids comprises ribosomal RNA (rRNA) and messenger RNA (mRNA). Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

In some embodiments, target nucleic acids comprise degraded DNA. Degraded DNA may be referred to as low-quality DNA or highly degraded DNA. Degraded DNA may be highly fragmented, and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A). In some embodiments, target nucleic acids are derived from nicked double-stranded nucleic acid fragments. Nicked double-stranded nucleic acid fragments may be denatured (e.g., heat denatured) to generate single-stranded nucleic acid fragments.

Nucleic acid may be derived from one or more sources (e.g., biological sample, blood, cells (e.g., circulating cells, tumor cells, circulating tumor cells (CTCs), a single cell, a single circulating cell, a single tumor cell, a single circulating tumor cell (CTT)), serum, plasma, buffy coat, urine, lymphatic fluid, skin, hair, soil, and the like) by methods known in the art. Any suitable method can be used for isolating, extracting and/or purifying DNA from a biological sample (e.g., from blood or a blood product), non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column. In some instances, a lysis protocol includes certain procedures described in Dabney et al., Proceedings of the National Academy of Sciences 110, no. 39 (2013): 15758-15763.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In some embodiments, cell-free nucleic acid comprises cell-free fetal nucleic acid (e.g., cell-free fetal DNA). In certain aspects, cell-free nucleic acid comprises circulating cancer nucleic acid (e.g., cancer DNA). In certain aspects, cell-free nucleic acid comprises circulating tumor nucleic acid (e.g., tumor DNA). In some embodiments, cell-free nucleic acid comprises infectious agent nucleic acid (e.g., pathogen DNA). In some embodiments, cell-free nucleic acid comprises nucleic acid (e.g., DNA) from a transplant. In some embodiments, cell-free nucleic acid comprises nucleic acid (e.g., DNA) from a microbiome (e.g., microbiome of gut, microbiome of blood, microbiome of mouth, microbiome of spinal fluid, microbiome of feces).

Cell-free DNA (cfDNA) may originate from degraded sources and often provides limiting amounts of DNA when extracted. cfDNA from cancer samples, for example, tends to have a higher population of short fragments. In certain instances, short fragments in cfDNA may be enriched for fragments originating from transcription factors rather than nucleosomes.

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In another example, blood serum or plasma from a pregnant female can include maternal nucleic acid and fetal nucleic acid. In another example, blood serum or plasma from a patient having an infection or infectious disease can include host nucleic acid and infectious agent or pathogen nucleic acid. In another example, a sample from a subject having received a transplant can include host nucleic acid and nucleic acid from the donor organ or tissue. In some instances, cancer nucleic acid, tumor nucleic acid, fetal nucleic acid, pathogen nucleic acid, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, tumor, fetal, pathogen, transplant, or microbiome nucleic acid). In another example, heterogeneous nucleic acid may include nucleic acid from two or more subjects (e.g., a sample from a crime scene).

At least two different nucleic acid species can exist in different amounts in extracellular nucleic acid and sometimes are referred to as minority species and majority species. In certain instances, a minority species of nucleic acid is from an affected cell type (e.g., cancer cell, wasting cell, cell attacked by immune system). In certain embodiments, a genetic variation or genetic alteration (e.g., copy number alteration, copy number variation, single nucleotide alteration, single nucleotide variation, chromosome alteration, and/or translocation) is determined for a minority nucleic acid species. In certain embodiments, a genetic variation or genetic alteration is determined for a majority nucleic acid species. Generally, it is not intended that the terms “minority” or “majority” be rigidly defined in any respect. In one aspect, a nucleic acid that is considered “minority,” for example, can have an abundance of at least about 0.1% of the total nucleic acid in a sample to less than 50% of the total nucleic acid in a sample. In some embodiments, a minority nucleic acid can have an abundance of at least about 1% of the total nucleic acid in a sample to about 40% of the total nucleic acid in a sample. In some embodiments, a minority nucleic acid can have an abundance of at least about 2% of the total nucleic acid in a sample to about 30% of the total nucleic acid in a sample. In some embodiments, a minority nucleic acid can have an abundance of at least about 3% of the total nucleic acid in a sample to about 25% of the total nucleic acid in a sample. For example, a minority nucleic acid can have an abundance of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% of the total nucleic acid in a sample. In some instances, a minority species of extracellular nucleic acid sometimes is about 1% to about 40% of the overall nucleic acid (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the nucleic acid is minority species nucleic acid). In some embodiments, the minority nucleic acid is extracellular DNA. In some embodiments, the minority nucleic acid is extracellular DNA from apoptotic tissue. In some embodiments, the minority nucleic acid is extracellular DNA from tissue where some cells therein underwent apoptosis. In some embodiments, the minority nucleic acid is extracellular DNA from necrotic tissue. In some embodiments, the minority nucleic acid is extracellular DNA from tissue where some cells therein underwent necrosis. Necrosis may refer to a post-mortem process following cell death, in certain instances. In some embodiments, the minority nucleic acid is extracellular DNA from tissue affected by a cell proliferative disorder (e.g., cancer). In some embodiments, the minority nucleic acid is extracellular DNA from a tumor cell. In some embodiments, the minority nucleic acid is extracellular fetal DNA. In some embodiments, the minority nucleic acid is extracellular DNA from a pathogen. In some embodiments, the minority nucleic acid is extracellular DNA from a transplant. In some embodiments, the minority nucleic acid is extracellular DNA from a microbiome.

In another aspect, a nucleic acid that is considered “majority,” for example, can have an abundance greater than 50% of the total nucleic acid in a sample to about 99.9% of the total nucleic acid in a sample. In some embodiments, a majority nucleic acid can have an abundance of at least about 60% of the total nucleic acid in a sample to about 99% of the total nucleic acid in a sample. In some embodiments, a majority nucleic acid can have an abundance of at least about 70% of the total nucleic acid in a sample to about 98% of the total nucleic acid in a sample. In some embodiments, a majority nucleic acid can have an abundance of at least about 75% of the total nucleic acid in a sample to about 97% of the total nucleic acid in a sample. For example, a majority nucleic acid can have an abundance of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total nucleic acid in a sample. In some embodiments, the majority nucleic acid is extracellular DNA. In some embodiments, the majority nucleic acid is extracellular maternal DNA. In some embodiments, the majority nucleic acid is DNA from healthy tissue. In some embodiments, the majority nucleic acid is DNA from non-tumor cells. In some embodiments, the majority nucleic acid is DNA from host cells.

In some embodiments, a minority species of extracellular nucleic acid is of a length of about 500 base pairs or less (e.g., about 80, 85, 90, 91, 92, 93, 94, 95, 96, 97, 98, 99 or 100% of minority species nucleic acid is of a length of about 500 base pairs or less). In some embodiments, a minority species of extracellular nucleic acid is of a length of about 300 base pairs or less (e.g., about 80, 85, 90, 91, 92, 93, 94, 95, 96, 97, 98, 99 or 100% of minority species nucleic acid is of a length of about 300 base pairs or less). In some embodiments, a minority species of extracellular nucleic acid is of a length of about 250 base pairs or less (e.g., about 80, 85, 90, 91, 92, 93, 94, 95, 96, 97, 98, 99 or 100% of minority species nucleic acid is of a length of about 250 base pairs or less). In some embodiments, a minority species of extracellular nucleic acid is of a length of about 200 base pairs or less (e.g., about 80, 85, 90, 91, 92, 93, 94, 95, 96, 97, 98, 99 or 100% of minority species nucleic acid is of a length of about 200 base pairs or less). In some embodiments, a minority species of extracellular nucleic acid is of a length of about 150 base pairs or less (e.g., about 80, 85, 90, 91, 92, 93, 94, 95, 96, 97, 98, 99 or 100% of minority species nucleic acid is of a length of about 150 base pairs or less). In some embodiments, a minority species of extracellular nucleic acid is of a length of about 100 base pairs or less (e.g., about 80, 85, 90, 91, 92, 93, 94, 95, 96, 97, 98, 99 or 100% of minority species nucleic acid is of a length of about 100 base pairs or less). In some embodiments, a minority species of extracellular nucleic acid is of a length of about 50 base pairs or less (e.g., about 80, 85, 90, 91, 92, 93, 94, 95, 96, 97, 98, 99 or 100% of minority species nucleic acid is of a length of about 50 base pairs or less).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, fetal nucleic acid can be purified from a mixture comprising maternal and fetal nucleic acid. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, small fragments of fetal nucleic acid (e.g., 30 to 500 bp fragments) can be purified, or partially purified, from a mixture comprising both fetal and maternal nucleic acid fragments. In certain examples, nucleosomes comprising smaller fragments of fetal nucleic acid can be purified from a mixture of larger nucleosome complexes comprising larger fragments of maternal nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

Nucleic acids may be amplified under amplification conditions. The term “amplified” or “amplification” or “amplification conditions” as used herein refers to subjecting a target nucleic acid in a sample or a nucleic acid product generated by a method herein to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). In certain instances, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule).

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any suitable form useful for conducting a sequence analysis.

In some embodiments, target nucleic acids are not modified in prior to combining with sequencing and/or library adapters. In some embodiments, target nucleic acids are not modified in length prior to combining with sequencing and/or library adapters. In this context, “not modified” means that target nucleic acids are isolated from a sample and then combined with sequencing and/or library adapters without modifying the length or the composition of the target nucleic acids. For example, target nucleic acids may not be shortened (e.g., they are not contacted with a restriction enzyme or nuclease or physical condition that reduces length (e.g., shearing condition, cleavage condition)) and may not be increased in length by one or more nucleotides (e.g., ends are not filled in at overhangs; no nucleotides are added to the ends). Adding a phosphate or chemically reactive group to one or both ends of a target nucleic acid generally is not considered modifying the nucleic acid or modifying the length of the nucleic acid. Denaturing a double-stranded nucleic acid (dsNA) fragment to generate a single-stranded nucleic acid (ssNA) fragment generally is not considered modifying the nucleic acid or modifying the length of the nucleic acid.

In some embodiments, one or both native ends of target nucleic acids are present when the nucleic acid is combined with sequencing and/or library adapters. Native ends generally refer to unmodified ends of a nucleic acid fragment. In some embodiments, native ends of target nucleic acids are not modified in length prior to combining with sequencing and/or library adapters. In this context, “not modified” means that target nucleic acids are isolated from a sample and then combined with sequencing and/or library adapters without modifying the length of the native ends of target nucleic acids. For example, target nucleic acids are not shortened (e.g., they are not contacted with a restriction enzyme or nuclease or physical condition that reduces length (e.g., shearing condition, cleavage condition) to generate non-native ends) and are not increased in length by one or more nucleotides (e.g., native ends are not filled in at overhangs; no nucleotides are added to the native ends). Adding a phosphate or chemically reactive group to one or both native ends of a target nucleic acid generally is not considered modifying the length of the nucleic acid.

In some embodiments, target nucleic acids are not contacting with a cleavage agent (e.g., endonuclease, exonuclease, restriction enzyme) and/or a polymerase prior to combining with sequencing and/or library adapters. In some embodiments, target nucleic acids are not subjected to mechanical shearing (e.g., ultrasonication (e.g., Adaptive Focused Acoustics™ (AFA) process by Covaris)) prior to combining with sequencing and/or library adapters. In some embodiments, target nucleic acids are not contacting with an exonuclease (e.g., DNAse) prior to combining with sequencing and/or library adapters. In some embodiments, target nucleic acids are not amplified prior to combining with sequencing and/or library adapters. In some embodiments, target nucleic acids are not attached to a solid support prior to combining with sequencing and/or library adapters. In some embodiments, target nucleic acids are not conjugated to another molecule prior to combining with sequencing and/or library adapters. In some embodiments, target nucleic acids are not cloned into a vector prior to combining with sequencing and/or library adapters. In some embodiments, target nucleic acids may be subjected to dephosphorylation prior to combining with sequencing and/or library adapters. In some embodiments, target nucleic acids may be subjected to phosphorylation prior to combining with sequencing and/or library adapters.

Overhangs

Target nucleic acids may comprise an overhang (e.g., at end of a nucleic acid fragment) and may comprise two overhangs (e.g., at both ends of a nucleic acid fragment). Nucleic acid overhangs can comprise different overhang lengths, and/or different overhang types (e.g., 5′ overhangs, 3′ overhangs, no overhangs). Target nucleic acids may comprise two overhangs, one overhang and one blunt end, two blunt ends, or a combination of these. Target nucleic acids may comprise two 3′ overhangs, two 5′ overhangs, one 3′ overhang and one 5′ overhang, one 3′ overhang and one blunt end, one 5′ overhang and one blunt end, two blunt ends, or a combination of these. In some cases, overhangs in double-stranded nucleic acids can be extended (i.e., filled in) prior to further processing (e.g., prior to denaturing).

In some embodiments, overhangs in target nucleic acids are native overhangs. In some embodiments, overhangs in target nucleic acids prior to extension are native overhangs. In some embodiments, target nucleic acid ends are native blunt ends. Native overhangs and native blunt ends generally refer to overhangs and blunt ends that have not been modified (e.g., have not been extended, have not been filled in, have not been cleaved or digested (e.g., by an endonuclease or exonuclease), have not been added or added to) prior to extension, prior to denaturation, and/or prior to combining with sequencing and/or library adapters. Often, native overhangs and native blunt ends generally refer to overhangs and blunt ends that have not been modified ex vivo (e.g., have not been extended in ex vivo, have not been filled in ex vivo, have not been cleaved or digested ex vivo (e.g., by an endonuclease or exonuclease), have not been added or added to ex vivo) prior to extension, prior to denaturation, and/or prior to combining with sequencing and/or library adapters. In certain instances, native overhangs and native blunt ends generally refer to overhangs and blunt ends that have not been modified after collection from a subject or source (e.g., have not been extended after collection from a subject or source, have not been filled in after collection from a subject or source, have not been cleaved or digested after collection from a subject or source (e.g., by an endonuclease or exonuclease), have not been added or added to after collection from a subject or source) prior to extension, prior to denaturation, and/or prior to combining with sequencing and/or library adapters. Native overhangs and native blunt ends generally do not include overhangs/ends created by contacting an isolated sample with a cleavage agent (e.g., endonuclease, exonuclease, restriction enzyme), and/or a polymerase. Native overhangs and native blunt ends generally do not include overhangs/ends created by mechanical shearing (e.g., ultrasonication (e.g., Adaptive Focused Acoustics™ (AFA) process by Covaris)). Native overhangs and native blunt ends generally do not include overhangs/ends created by contacting an isolated sample with an exonuclease (e.g., DNAse). Native overhangs and native blunt ends generally do not include overhangs/ends created by amplification (e.g., polymerase chain reaction). Native overhangs and native blunt ends generally do not include overhangs/ends attached to a solid support, conjugated to another molecule, or cloned into a vector. In some embodiments, native overhangs and native blunt ends may be subjected to dephosphorylation and may be referred to as dephosphorylated native overhangs and dephosphorylated native blunt ends. In some embodiments, native overhangs and native blunt ends may be subjected to phosphorylation and may be referred to as phosphorylated native overhangs and phosphorylated native blunt ends.

Some or all target nucleic acids may comprise double-stranded nucleic acid (dsNA) comprising an overhang. Some or all target nucleic acids may comprise double-stranded DNA (dsDNA) comprising an overhang. Target nucleic acids comprising an overhang may comprise a duplex region and a single-stranded overhang. A target nucleic acid having at least one overhang may be extended such that the overhang is filed in and a blunt end is generated. An extended target nucleic acid may comprise an extension region complementary to an overhang (i.e., an overhang present in the target nucleic acid prior to extension).

Nucleic Acid Library

Methods herein may include preparing a nucleic acid library and/or modifying nucleic acids for a nucleic acid library. In some embodiments, ends of nucleic acid fragments are modified such that the fragments, or amplified products thereof, may be incorporated into a nucleic acid library. Generally, a nucleic acid library refers to a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI), a palindromic sequence described herein, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters (e.g., oligonucleotide adapters described in PCT Patent Publication No. WO 2019/140201; scaffold adapters described in PCT Patent Publication No. WO 2020/206143). In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation (e.g., when using cell-free DNA).

In certain embodiments, a ligation-based library preparation method is used (e.g., ILLUMINA TRUSEQ, Illumina, San Diego CA). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides.

In some embodiments, end repair is omitted and scaffold adapters (e.g., scaffold adapters described in PCT Patent Publication No. WO 2020/206143) are hybridized and ligated directly to the native ends of nucleic acids (e.g., single-stranded nucleic acids, fragmented nucleic acids, and/or cell-free DNA). Scaffold adapters may comprise an oligonucleotide adapter and a scaffold polynucleotide where the scaffold polynucleotide comprises a single-stranded nucleic acid (ssNA) hybridization region and an oligonucleotide hybridization region. A nucleic acid composition (e.g., a nucleic acid sample), the oligonucleotide, and the scaffold polynucleotide may be combined under conditions in which the scaffold polynucleotide is hybridized to (i) an ssNA terminal region and (ii) the oligonucleotide, thereby forming hybridization products in which an end of the oligonucleotide is adjacent to an end of the ssNA terminal region.

In some embodiments, end repair is omitted and a pool of oligonucleotide adapters (e.g., oligonucleotide adapters described in PCT Patent Publication No. WO 2019/140201) are hybridized and/or ligated directly to the native ends of nucleic acids (e.g., double-stranded nucleic acids, double-stranded nucleic acids having at least one overhang, blunt-ended double-stranded nucleic acids, fragmented nucleic acids, and/or cell-free DNA). Some or all adapters in a pool of oligonucleotide adapters may comprise two strands, and an overhang at a first end and, in certain configurations, two non-complementary strands at a second end, where the overhang is capable of hybridizing to a target nucleic acid overhang, where each oligonucleotide has a unique overhang sequence and length, and where each oligonucleotide comprises an oligonucleotide overhang identification sequence specific to one or more features of the oligonucleotide overhang. A nucleic acid composition (e.g., a nucleic acid sample) and a pool of oligonucleotide adapters may be combined under conditions in which oligonucleotide adapter overhangs hybridize to target nucleic acid overhangs having a corresponding length, thereby forming hybridization products.

In some embodiments, nucleic acid library preparation comprises ligating an oligonucleotide adapter, a scaffold adapter, or component thereof, (e.g., to a sample nucleic acid, to a sample nucleic acid fragment, to a template nucleic acid, to a target nucleic acid, to an ssNA). Oligonucleotide adapters, scaffold adapters, or components thereof, may comprise sequences complementary to flow-cell anchors, and sometimes are utilized to immobilize a nucleic acid library to a solid support, such as the inside surface of a flow cell, for example. In some embodiments, an oligonucleotide adapter, a scaffold adapter, or component thereof, comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, an oligonucleotide adapter, a scaffold adapter, or component thereof, comprises one or more of primer annealing polynucleotide, also referred to herein as priming sequence or primer binding domain, (e.g., for annealing to flow cell attached oligonucleotides and/or to free amplification primers), an index polynucleotide (e.g., sample index sequence for tracking nucleic acid from different samples; also referred to as a sample ID), a barcode polynucleotide (e.g., single molecule barcode (SMB) for tracking individual molecules of sample nucleic acid that are amplified prior to sequencing; also referred to as a molecular barcode or a unique molecular identifier (UMI)). In some embodiments, a primer annealing component (or priming sequence or primer binding domain) of an oligonucleotide adapter, a scaffold adapter, or component thereof, comprises one or more universal sequences (e.g., sequences complementary to one or more universal amplification primers). In some embodiments, an index polynucleotide (e.g., sample index; sample ID) is a component of an oligonucleotide adapter, a scaffold adapter, or component thereof. In some embodiments, an index polynucleotide (e.g., sample index; sample ID) is a component of a universal amplification primer sequence.

In some embodiments, oligonucleotide adapters, scaffold adapters, or components thereof, when used in combination with amplification primers (e.g., universal amplification primers) are designed generate library constructs comprising one or more of: universal sequences, molecular barcodes (UMIs), UMI flanking sequence, sample ID sequences, spacer sequences, and a sample nucleic acid sequence. In some embodiments, oligonucleotide adapters, scaffold adapters, or components thereof, when used in combination with universal amplification primers are designed to generate library constructs comprising an ordered combination of one or more of: universal sequences, molecular barcodes (UMIs), sample ID sequences, spacer sequences, and a sample nucleic acid sequence. For example, a library construct may comprise a first universal sequence, followed by a second universal sequence, followed by first molecular barcode (UMI), followed by a spacer sequence, followed by a template sequence (e.g., sample nucleic acid sequence), followed by a spacer sequence, followed by a second molecular barcode (UMI), followed by a third universal sequence, followed by a sample ID, followed by a fourth universal sequence. In some embodiments, oligonucleotide adapters, scaffold adapters, or components thereof, when used in combination with amplification primers (e.g., universal amplification primers) are designed generate library constructs for each strand of a template molecule (e.g., sample nucleic acid molecule). In some embodiments, oligonucleotide adapters and/or scaffold adapters are duplex adapters.

An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). In some embodiments, an identifier is incorporated into or attached to a nucleic acid prior to a sequencing method (e.g., by an extension reaction, by an amplification reaction, by a ligation reaction). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, an identifier, a sequencing-specific index/barcode, and a sequencer-specific flow-cell binding primer sites are incorporated into a nucleic acid library by single-primer extension (e.g., by a strand displacing polymerase).

In some embodiments, a nucleic acid library or parts thereof are amplified (e.g., amplified by a PCR-based method) under amplification conditions. In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Application Publication No. 2013/0012399), the like or combinations thereof.

Nucleic acid sequencing In some embodiments, nucleic acid (e.g., nucleic acid fragments, sample nucleic acid, cell-free nucleic acid, single-stranded nucleic acid, single-stranded DNA, single-stranded RNA) is sequenced. In some embodiments, the sequencing process generates sequence reads (or sequencing reads). In some embodiments, a method herein comprises determining the sequence of a nucleic acid molecule based on the sequence reads. In some embodiments, a sequencing process herein comprises whole genome sequencing. In some embodiments, a sequencing process herein comprises genome-wide sequencing. In some embodiments, a sequencing process herein generates thousands to millions of sequence reads.

For certain sequencing platforms (e.g., paired-end sequencing), generating sequence reads may include generating forward sequence reads and generating reverse sequence reads. For example, sequencing using certain paired-end sequencing platforms sequence each nucleic acid fragment from both directions, generally resulting in two reads per nucleic acid fragment, with the first read in a forward orientation (forward read) and the second read in reverse-complement orientation (reverse read). For certain platforms, a forward read is generated off a particular primer within a sequencing adapter (e.g., ILLUMINA adapter, P5 primer), and a reverse read is generated off a different primer within a sequencing adapter (e.g., ILLUMINA adapter, P7 primer).

Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeg™, MiSeg™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLiD sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

The length of a sequence read is often associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp.

In some embodiments. the nominal, average, mean or absolute length of single-end reads sometimes is about 10 continuous nucleotides to about 250 or more contiguous nucleotides, about 15 contiguous nucleotides to about 200 or more contiguous nucleotides, about 15 contiguous nucleotides to about 150 or more contiguous nucleotides, about 15 contiguous nucleotides to about 125 or more contiguous nucleotides, about 15 contiguous nucleotides to about 100 or more contiguous nucleotides, about 15 contiguous nucleotides to about 75 or more contiguous nucleotides, about 15 contiguous nucleotides to about 60 or more contiguous nucleotides, 15 contiguous nucleotides to about 50 or more contiguous nucleotides, about 15 contiguous nucleotides to about 40 or more contiguous nucleotides, and sometimes about 15 contiguous nucleotides or about 36 or more contiguous nucleotides. In certain embodiments the nominal, average, mean or absolute length of single-end reads is about 20 to about 30 bases, or about 24 to about 28 bases in length. In certain embodiments the nominal, average, mean or absolute length of single-end reads is about 1, 2, 3, 4, 5, 6, 7, 8, 9, 10, 11, 12, 13, 14, 15, 16, 17, 18, 19, 21, 22, 23, 24, 25, 26, 27, 28 or about 29 bases or more in length. In certain embodiments the nominal, average, mean or absolute length of single-end reads is about 20 to about 200 bases, about 100 to about 200 bases, or about 140 to about 160 bases in length. In certain embodiments the nominal, average, mean or absolute length of single-end reads is about 30, 40, 50, 60, 70, 80, 90, 100, 110, 120, 130, 140, 150, 160, 170, 180, 190, or about 200 bases or more in length. In certain embodiments, the nominal, average, mean or absolute length of paired-end reads sometimes is about 10 contiguous nucleotides to about 25 contiguous nucleotides or more (e.g., about 10, 11, 12, 13, 14, 15, 16, 17, 18, 19, 20, 21, 22, 23, 24 or 25 nucleotides in length or more), about 15 contiguous nucleotides to about 20 contiguous nucleotides or more, and sometimes is about 17 contiguous nucleotides or about 18 contiguous nucleotides. In certain embodiments, the nominal, average, mean or absolute length of paired-end reads sometimes is about 25 contiguous nucleotides to about 400 contiguous nucleotides or more (e.g., about 25, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides in length or more), about 50 contiguous nucleotides to about 350 contiguous nucleotides or more, about 100 contiguous nucleotides to about 325 contiguous nucleotides, about 150 contiguous nucleotides to about 325 contiguous nucleotides, about 200 contiguous nucleotides to about 325 contiguous nucleotides, about 275 contiguous nucleotides to about 310 contiguous nucleotides, about 100 contiguous nucleotides to about 200 contiguous nucleotides, about 100 contiguous nucleotides to about 175 contiguous nucleotides, about 125 contiguous nucleotides to about 175 contiguous nucleotides, and sometimes is about 140 contiguous nucleotides to about 160 contiguous nucleotides. In certain embodiments, the nominal, average, mean, or absolute length of paired-end reads is about 150 contiguous nucleotides, and sometimes is 150 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample from a subject can be reads from a mixture of a minority nucleic acid and a majority nucleic acid. For example, sequence reads obtained from the blood of a cancer patient can be reads from a mixture of cancer nucleic acid and non-cancer nucleic acid. In another example, sequence reads obtained from the blood of a pregnant female can be reads from a mixture of fetal nucleic acid and maternal nucleic acid. In another example, sequence reads obtained from the blood of a patient having an infection or infectious disease can be reads from a mixture of host nucleic acid and pathogen nucleic acid. In another example, sequence reads obtained from the blood of a transplant recipient can be reads from a mixture of host nucleic acid and transplant nucleic acid. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces) in a subject. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces), and nucleic acid from the host subject. A mixture of relatively short reads can be transformed by processes described herein into a representation of genomic nucleic acid present in the subject, and/or a representation of genomic nucleic acid present in a tumor, a fetus, a pathogen, a transplant, or a microbiome.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments, specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, a genome (e.g., a whole genome) is sequenced with about 1-fold to about 200-fold coverage, or about 50-fold to 100-fold coverage (e.g., about 1-, 2-, 3-, 4-, 5-, 6-, 7-, 8-, 9-, 10-, 20-, 30-, 40-, 50-, 60-, 70-, 80-, 90-, 100-, 150-, 200-fold or greater coverage). In some embodiments, a genome (e.g., a whole genome) is sequenced with at least about 1-fold coverage. In some embodiments, a genome (e.g., a whole genome) is sequenced with at least about 2-fold coverage. In some embodiments, a genome (e.g., a whole genome) is sequenced with about 10-fold coverage. In some embodiments, a genome (e.g., a whole genome) is sequenced with about 50-fold coverage. In some embodiments, a genome (e.g., a whole genome) is sequenced with about 100-fold coverage.

In some embodiments, a test sample is sequenced using low coverage sequencing. Low coverage sequencing may be referred to as shallow depth sequencing. Low coverage sequencing may refer to sequencing at about 10-fold coverage or less. In some embodiments, a test sample is sequenced at about 10-fold coverage or less. In some embodiments, a test sample is sequenced at about 9-fold coverage or less. In some embodiments, a test sample is sequenced at about 8-fold coverage or less. In some embodiments, a test sample is sequenced at about 7-fold coverage or less. In some embodiments, a test sample is sequenced at about 6-fold coverage or less. In some embodiments, a test sample is sequenced at about 5-fold coverage or less. In some embodiments, a test sample is sequenced at about 4-fold coverage or less. In some embodiments, a test sample is sequenced at about 3-fold coverage or less. In some embodiments, a test sample is sequenced at about 2-fold coverage or less. In some embodiments, a test sample is sequenced at about 1-fold coverage or less. In some embodiments, a test sample is sequenced at a fold coverage between about 0.5-fold to about 2-fold. In some embodiments, a test sample is sequenced at about 2-fold coverage. In some embodiments, a test sample is sequenced at about 1-fold coverage. In some embodiments, a test sample is sequenced at about 0.9-fold coverage or less. In some embodiments, a test sample is sequenced at about 0.8-fold coverage or less. In some embodiments, a test sample is sequenced at about 0.7-fold coverage or less. In some embodiments, a test sample is sequenced at about 0.6-fold coverage or less. In some embodiments, a test sample is sequenced at about 0.5-fold coverage or less.

In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used.

In some embodiments, a shotgun sequencing method is used. Shotgun sequencing generally refers to sequencing random nucleic acid strands. For example, DNA may be broken up randomly into numerous small fragments or DNA may be present as small fragments in a sample (e.g., cell-free DNA, degraded DNA). The DNA fragments are sequenced to obtain sequence reads. Multiple overlapping reads for the target DNA are obtained, and the overlapping reads are used to assemble the reads into a continuous sequence (typically performed using a computer program).

In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In certain embodiments, sequence reads are generated using a whole genome sequencing approach. In certain embodiments, sequence reads are generated using a genome-wide sequencing approach. In certain embodiments, sequence reads are generated using a massively parallel sequencing approach. In certain embodiments, sequence reads are generated by a non-targeted sequencing approach. In certain embodiments, sequence reads are generated using a genome-wide, massively parallel sequencing approach. In certain embodiments, sequence reads are generated using a non-targeted, genome-wide sequencing approach. In certain embodiments, sequence reads are generated using a non-targeted, massively parallel sequencing approach. In certain embodiments, sequence reads are generated using a non-targeted, genome-wide, massively parallel sequencing approach.

Whole genome, genome-wide, massively parallel, and/or non-targeted sequencing approaches generate massive amounts of data. The human genome is approximately 3 billion base pairs in size. An example sequencing process performed on a test sample at 1-fold coverage would generate at least 3 million 1 kb reads. Sequencing processes that produce smaller reads and/or are performed at greater than 1-fold coverage would generate more than 3 million reads. Accordingly, such sequence data typically is processed (e.g., aligned, analyzed for alleles at target and linked loci, quantified, assessed for genotypes) using a computer, as the sheer volume of such data makes it impractical or impossible for a human to perform such a task without the use of a computer and/or software. In some embodiments, a method herein comprises generating, obtaining, and/or processing at least 100,000 sequence reads. In some embodiments, a method herein comprises generating, obtaining, and/or processing at least 500,000 sequence reads. In some embodiments, a method herein comprises generating, obtaining, and/or processing at least 1,000,000 sequence reads. In some embodiments, a method herein comprises generating, obtaining, and/or processing at least 2,000,000 sequence reads. In some embodiments, a method herein comprises generating, obtaining, and/or processing at least 3,000,000 sequence reads.

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (IIlumina, San Diego CA)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include ILLUMINA/SOLEX/HISEQ (e.g., Illumina's Genome Analyzer; Genome Analyzer II; HISEQ 2000; HISEQ), SOLiD, Roche/454, PACBIO and/or SMRT, Helicos True Single Molecule Sequencing, Ion Torrent and Ion semiconductor-based sequencing (e.g., as developed by Life Technologies), WildFire, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, nucleic acid is sequenced and the sequencing product (e.g., a collection of sequence reads) is processed prior to, or in conjunction with, an analysis of the sequenced nucleic acid. For example, sequence reads may be processed according to one or more of the following: aligning, mapping, filtering, counting, normalizing, weighting, generating a profile, and the like, and combinations thereof. Certain processing steps may be performed in any order and certain processing steps may be repeated.

Machines, Software and Interfaces

Certain processes and methods described herein often are too complex for performing in the mind and cannot be performed without a computer, microprocessor, software, module or other machine. Methods described herein may be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces may be used to conduct methods described herein. Using machines, software and interfaces, a user may enter, request, query or determine options for using particular information, programs or processes, which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read processing; send processed sequence read data to a computer system for further processing and/or yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine an outcome from the sequence reads (e.g., a characteristic of a sample).

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like.

Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, sequence read information may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data) and may include a module that specifically processes the data (e.g., a processing module that processes received data. The terms “obtaining” and “receiving” input information refers to receiving data (e.g., sequence reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

Provided herein, in certain embodiments, are systems, machines and apparatuses comprising one or more microprocessors and memory, which memory comprises instructions executable by the one or more microprocessors and which instructions executable by the one or more microprocessors are configured to (1) obtain sequence reads, which sequence reads are of sample DNA from a subject, the sample DNA comprising circulating tumor DNA (ctDNA) and cell-free DNA (cfDNA); (2) detect at least one ctDNA property of (i) a patient-specific ctDNA property and (ii) a general ctDNA property; and (3) determine at least some of the sequencing reads as ctDNA sequencing reads based on the at least one ctDNA property.

Provided herein, in certain embodiments, are machines comprising one or more microprocessors and memory, which memory comprises instructions executable by the one or more microprocessors and which memory comprises sequence reads, which sequence reads are of sample DNA from a subject, the sample DNA comprising circulating tumor DNA (ctDNA) and cell-free DNA (cfDNA), and which instructions executable by the one or more microprocessors are configured to (1) detect at least one ctDNA property of (i) a patient-specific ctDNA property and (ii) a general ctDNA property; and (2) determine at least some of the sequencing reads as ctDNA sequencing reads based on the at least one ctDNA property.

Provided herein, in certain embodiments, are non-transitory computer-readable storage media with an executable program stored thereon, where the program instructs a microprocessor to perform the following: (1) access sequence reads, which sequence reads are of sample DNA from a subject, the sample DNA comprising circulating tumor DNA (ctDNA) and cell-free DNA (cfDNA), (2) detect at least one ctDNA property of (i) a patient-specific ctDNA property and (ii) a general ctDNA property; and (3) determine at least some of the sequencing reads as ctDNA sequencing reads based on the at least one ctDNA property.

Provided herein, in certain embodiments, are systems, machines and apparatuses comprising one or more microprocessors and memory, which memory comprises instructions executable by the one or more microprocessors and which instructions executable by the one or more microprocessors are configured to (1) obtain sequence reads, which sequence reads are of sample DNA from a subject, the sample DNA comprising circulating tumor DNA (ctDNA) and cell-free DNA (cfDNA); (2) detect (i) one or more ctDNA properties of a patient-specific ctDNA property and/or (ii) one or more ctDNA properties of a general ctDNA property; and (3) determine at least some of the sequencing reads as ctDNA sequencing reads based on the one or more ctDNA properties.

Provided herein, in certain embodiments, are machines comprising one or more microprocessors and memory, which memory comprises instructions executable by the one or more microprocessors and which memory comprises sequence reads, which sequence reads are of sample DNA from a subject, the sample DNA comprising circulating tumor DNA (ctDNA) and cell-free DNA (cfDNA), and which instructions executable by the one or more microprocessors are configured to (1) detect (i) one or more ctDNA properties of a patient-specific ctDNA property and/or (ii) one or more ctDNA properties of a general ctDNA property; and (2) determine at least some of the sequencing reads as ctDNA sequencing reads based on the one or more ctDNA properties.

Provided herein, in certain embodiments, are non-transitory computer-readable storage media with an executable program stored thereon, where the program instructs a microprocessor to perform the following: (1) access sequence reads, which sequence reads are of sample DNA from a subject, the sample DNA comprising circulating tumor DNA (ctDNA) and cell-free DNA (cfDNA), (2) detect (i) one or more ctDNA properties of a patient-specific ctDNA property and/or (ii) one or more ctDNA properties of a general ctDNA property; and (3) determine at least some of the sequencing reads as ctDNA sequencing reads based on the one or more ctDNA properties.

Certain Implementations

Following are non-limiting examples of certain implementations of the technology.

A1. A method, comprising:
obtaining a sample from a subject, the sample comprising DNA comprising circulating tumor DNA (ctDNA) and cell-free DNA (cfDNA);
sequencing the DNA to generate sequencing reads;
detecting at least one ctDNA property of (i) a patient-specific ctDNA property and (ii) a general ctDNA property; and
determining at least some of the sequencing reads as ctDNA sequencing reads based on the at least one ctDNA property.

A1.1 A method, comprising:
obtaining sequence reads, which sequence reads are of sample DNA from a subject, the sample DNA comprising circulating tumor DNA (ctDNA) and cell-free DNA (cfDNA);
detecting at least one ctDNA property of (i) a patient-specific ctDNA property and (ii) a general ctDNA property; and
determining at least some of the sequencing reads as ctDNA sequencing reads based on the at least one ctDNA property.

A1.2 A method, comprising:
obtaining a sample from a subject, the sample comprising DNA comprising circulating tumor DNA (ctDNA) and cell-free DNA (cfDNA);
sequencing the DNA to generate sequencing reads;
detecting (i) one or more ctDNA properties of a patient-specific ctDNA property and/or (ii) one or more ctDNA properties of a general ctDNA property; and
determining at least some of the sequencing reads as ctDNA sequencing reads based on the one or more ctDNA properties.

A1.3 A method, comprising:
obtaining sequence reads, which sequence reads are of sample DNA from a subject, the sample DNA comprising circulating tumor DNA (ctDNA) and cell-free DNA (cfDNA);
detecting (i) one or more ctDNA properties of a patient-specific ctDNA property and/or (ii) one or more ctDNA properties of a general ctDNA property; and
determining at least some of the sequencing reads as ctDNA sequencing reads based on the one or more ctDNA properties.

A1.4 A method, comprising:
a) obtaining a first sample from a subject, the first sample comprising DNA comprising circulating tumor DNA (ctDNA);
b) obtaining a second sample from a subject, the second sample comprising DNA comprising circulating tumor DNA (ctDNA) and cell-free DNA (cfDNA);
c) sequencing the DNA obtained in (a) and (b) to generate sequencing reads;
d) detecting at least one ctDNA property of (i) a patient-specific ctDNA property and (ii) a general ctDNA property; and
e) determining at least some of the sequencing reads as ctDNA sequencing reads based on the at least one ctDNA property.

A1.5 A method, comprising:
obtaining sequence reads, which sequence reads are of a) a first sample DNA from a subject, the first sample DNA comprising circulating tumor DNA (ctDNA), and b) a second sample DNA from a subject, the second sample DNA comprising circulating tumor DNA (ctDNA) and cell-free DNA (cfDNA);
detecting at least one ctDNA property of (i) a patient-specific ctDNA property and (ii) a general ctDNA property; and
determining at least some of the sequencing reads as ctDNA sequencing reads based on the at least one ctDNA property.

A1.6 The method of embodiment A1.4 or A1.5, wherein the first sample comprises a single circulating tumor cell (CTC) or a plurality of CTCs.

A1.7. The method of any one of embodiments A1.4 to A1.6, wherein the second sample comprises blood, serum, or plasma.

A2. The method of any one of embodiments A1-A1.7, wherein the at least one ctDNA property comprises a mutation.

A3. The method of embodiment A2, wherein the mutation is a tumor somatic mutation.

A4. The method of any one of embodiments A1-A3, wherein the at least one ctDNA property comprises genomic DNA accessibility.

A5. The method of embodiment A4, wherein the genomic DNA accessibility is a differential genomic DNA accessibility compared to an expectation set of genomic DNA accessibility.

A6. The method of any one of embodiments A1-A5, wherein the at least one ctDNA property comprises methylation.

A7. The method of any one of embodiment A1-A6, wherein the at least one ctDNA property comprises a transcriptome profile.

A8. The method of any one of embodiments A1-A7, wherein the at least one ctDNA property comprises nucleosome positioning.

A9. The method of any one of embodiments A1-A8, wherein the at least one ctDNA property comprises chromatin structure.

A10. The method of any one of embodiments A1-A9, wherein the at least one ctDNA property comprises 3D nucleus organization of a nucleus.

A11. The method of any one of embodiments A1-A10, wherein the at least one ctDNA property comprises a copy number variation. A12. The method of any one of embodiment A1-A11, wherein the at least one ctDNA property comprises expression levels of one or more genes.

A13. The method of embodiment A12, wherein a gene of the one or more genes encodes a nuclease.

A14. The method of embodiment A12, wherein a gene of the one or more genes encodes an apoptosis pathway member.

A15. The method of embodiment A12, wherein a gene of the one or more genes encodes a necrosis pathway member.

A16. The method of any one of embodiments A1-A15, wherein the at least one ctDNA property comprises base composition of nucleic acid fragment native ends.

A17. The method of any one of embodiments A1-A16, wherein the at least one ctDNA property comprises genomic context of nucleic acid fragment native ends.

A18. The method of any one of embodiments A1-A17, wherein the at least one ctDNA property comprises read depth coverage at one or more loci.

A19. The method of any one of embodiments A1-A18, wherein the at least one ctDNA property comprises epigenetic protein modification.

A20. The method of embodiment A19, wherein the epigenetic protein modification is histone methylation.

A21. The method of embodiment A19, wherein the epigenetic protein modification is histone acetylation.

A22. The method of embodiment A19, wherein the epigenetic protein modification is histone phosphorylation.

A23. The method of any one of embodiments A1-A22, wherein the at least one ctDNA property comprises fragment length.

A24. The method of any one of embodiments A1-A23, wherein the at least one ctDNA property comprises fragment overhang sequence.

A25. The method of any one of embodiments A1-A24, wherein the at least one ctDNA property comprises fragment overhang length.

A26. The method of any one of embodiments A1-A25, wherein the at least one ctDNA property comprises fragment overhang directionality.

A27. The method of any one of embodiments A1-A26, wherein the sequence reads are generated by a non-targeted sequencing process.

A28. The method of any one of embodiments A1-A27, wherein the sequence reads are generated by a genome-wide sequencing process.

A29. The method of any one of embodiments A1-A28, wherein the sequence reads are generated by a massively parallel sequencing process.

A30. The method of any one of embodiments A1-A29, wherein at least 100,000 sequence reads are generated.

### EXAMPLES

The examples set forth below illustrate certain implementations and do not limit the technology.

### Example 1: Library Preparations for Single and Multiple Cells

In the following example, a cell lysis and library preparation protocol was performed using human embryonic kidney (HEK) cells. The length distribution of fragments from the amplified library were as expected, and human DNA sequences were detected in libraries prepared from samples 1˜4 (described below) and no library or human sequences were detected from sample 5 (negative control). The protocol below may be used for preparing DNA sequencing libraries from circulating tumor cells and/or from a single circulating tumor cell.

Lyse Cells/Digest DNA:

Combine the following:
Sample 1: a single HEK cell (should be 2 μl)

Sample 2: a single HEK cell (should be 2 μl)
Sample 3: 1,000 HEK cells (from frozen)
Sample 4: 1,000 HEK cells (from frozen)
Sample 5: water (negative control)

Bring up the volume to 14 μl with:
1.5 μl of DNase I reaction buffer
1.5 μl of 1% Triton-X (stock solution is 10%, dilute to 1% in H₂O first)
Fill with H₂O, Pipette mix gingerly

Add the following:
Sample 1: 1 μl of NEB DNase I
Sample 2: 1 μl of 1:10 diluted NEB DNase I (dilute 1× DNase I reaction buffer)
Sample 3: 1 μl of NEB DNase I
Sample 4: 1 μl of 1:10 diluted NEB DNase I (dilute 1× DNase I reaction buffer)
Sample 5: 1 μl of NEB Dnase I

Incubate at 37° C. for 5 min.

Quickly add the following per reaction (make a master mix of everything and then add 14 μl per reaction):
3 μl of 100 mM Tris-HCl pH7.5 (if stock solution is 1M, 1:10 dilute to 100 mM with H₂O first)
3 μl of 50 mM EDTA (if stock solution is 500 mM, 1:10 dilute to 50 mM with H₂O first)
3 μl of 10% SDS stock solution
3 μl of 50 mM NaCl (if stock solution is 5 M, 1:100 dilute to 50 mM with H₂O first)
3 μl H₂O

Then add 1 μl NEB ProtK and place at 55° C. for 30 min to 1 hr.

SPRI Clean:

Bring sample up to 50 μl using H₂O.

Add 100 μl SPRI beads.
Perform SPRI clean.
Elute in 18 μl elution buffer (EB) and proceed to single-stranded library prep.

Single-Stranded Library Prep (SRSLY):

Denature:
1. Add 2 μl SSE
2. 2 min ice, 3 min 98° C., 2 min ice

Ligation:
1. Add 2 μl of 1×P5 PicoPlus Adapter (CLARETBIO, Santa Cruz, CA; i.e., scaffold adapter described herein and in WO 2020/206143)
2. Add 2 μl of 1×P7 PicoPlus Adapter (CLARETBIO, Santa Cruz, CA; i.e., scaffold adapter described herein and in WO 2020/206143)
3. Add 26 μl of SRSLY master mix (CLARETBIO, Santa Cruz, CA)
4. Mix vigorously by vortexing then spin down
5. Incubate reactions for 1 h at 37° C.

Cleaning SRSLY Reaction
1. Bead Cleans:
   a. Moderate Retention: add 60 μl EB and 65.2 μl SPRI beads
2. Perform SPRI clean protocol. Elute in 20 μl H₂O or EB

Indexing PCR
1. Combine 20 μl elution with 2.5 μl P5 index, 2.5 μl P7 index, 25 μl KAPA HiFi master mix
2. Run:
   a. 98° C. 3 min
   b. cycles of 98° C. 20 sec, 65° C. 30 sec, 72° C. 30 sec
      i. Run 18 cycles for the single cells and the control (samples 1, 2, 5)
      ii. Run 16 cycles for the 1000 cells (samples 3, 4)
   c. 72° C. 1 min
   d. 12° C. ∞

Final Clean
1. Bead Clean:
   a. Moderate Retention: add 60 μl SPRI beads
2. Perform SPRI protocol
   a. Elute in 20 μl low TE
      i. Qubit
      ii. Tapestation
Re-amplify and clean in certain instances.

### Example 2: Library Preparations for Single and Multiple Cells (Modified Protocol)

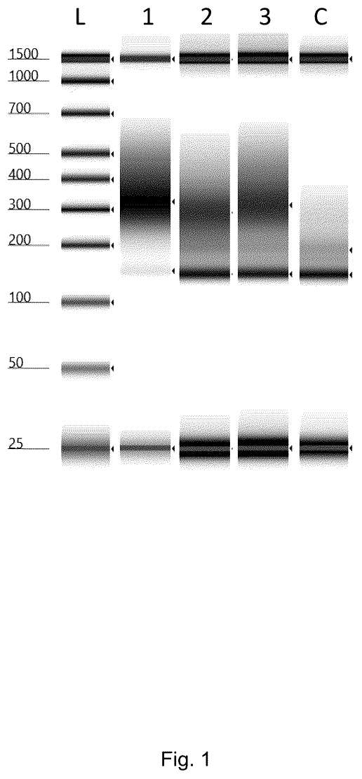

In the following example, a modified cell lysis and library preparation protocol was performed using human embryonic kidney (HEK) cells. The length distribution of fragments from the amplified library were as expected (FIG. 1), and human DNA sequences were detected in libraries prepared from the samples. The protocol below may be used for preparing DNA sequencing libraries from circulating tumor cells and/or from a single circulating tumor cell.

Lyse/Fragment DNA:
1. Bring cell(s) up to 14 μl in a solution of 1×PBS containing 0.1% TWEEN-20, 1×NEB DNase I buffer (Cat No. B0303S), 0.1% Triton-X final concentrations
2. In a separate tube dilute NEB DNase I (Cat No. M0303S) 1:100 using 1×DNase I buffer
3. Add 1 μl of the 1:100 diluted DNase I to the 14 μl containing the cell(s)
4. Incubate at 37° C. for 5 min.
5. Add 14 μl of stop solution to each tube.
   a. Stop solution is 20 mM Tris-HCL 7.5, 10 mM EDTA, 2% SDS, 10 mM NaCl, and 2% TWEEN-20
6. Add 1 μl of NEB Proteinase K to each tube (Cat No. P8107S)
7. Incubate samples at 55° C. for 45 minutes

Purify DNA:
1. Bring samples up to 50 μl using 1×PBS containing 0.1% TWEEN-20
2. Add 100 μl SPRI beads
   a. Perform SPRI clean
   b. Elute in 18 μl of 1×PBS containing 0.1% TWEEN-20

Single-Stranded Library Prep (SRSLY):
1. Add 2 μl SSE
2. 2 min ice, 3 min 98° C., 2 min ice
6. Add 2 μl of 1×P5 PicoPlus Adapter (CLARETBIO, Santa Cruz, CA; i.e., scaffold adapter described herein and in WO 2020/206143)
7. Add 2 μl of 1×P7 PicoPlus Adapter (CLAREBIO, Santa Cruz, CA; i.e., scaffold adapter described herein and in WO 2020/206143)
3. Add 26 μl of SRSLY master mix (CLARETBIO, Santa Cruz, CA)
4. Mix vigorously by vortexing then spin down
5. Incubate reactions for 1 h at 37° C.

Purify DNA
1. Add 60 μl 1×PBS containing 0.1% TWEEN-20 and 65.2 μl SPRI beads to the 50 μl SRSLY ligation reaction
   a. Perform SPRI clean protocol.
   b. Elute in 20 μl of 1×PBS containing 0.1% TWEEN-20

Indexing PCR
1. Combine 20 μl elution with 2.5 μl 20 OA P5 index, 2.5 μl 20 OA P7 index, and 25 μl 2×KAPA HiFi master mix
2. Run:
   1. 98° C. 3 min
   2. 17 cycles of 98° C. 20 sec, 65° C. 30 sec, 72° C. 30 sec
   3. 72° C. 1 min
   4. 12° C. ∞

Purify DNA
1. Add 60 μl SPRI beads to the 50 μl index PCR reaction
   a. Perform SPRI protocol
   b. Elute in 20 μl low TE
      i. Qubit
      ii. Tapestation

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the 5 listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

1. A method, comprising:
obtaining a sample from a subject, the sample comprising DNA comprising circulating tumor DNA (ctDNA) and cell-free DNA (cfDNA);
sequencing the DNA to generate sequencing reads;
detecting at least one ctDNA property of (i) a patient-specific ctDNA property and (ii) a general ctDNA property; and
determining at least some of the sequencing reads as ctDNA sequencing reads based on the at least one ctDNA property.

2. The method of claim 1, wherein the at least one ctDNA property comprises a mutation.

3. The method of claim 2, wherein the mutation is a tumor somatic mutation.

4. The method of claim 1, wherein the at least one ctDNA property comprises genomic DNA accessibility.

5. The method of claim 4, wherein the genomic DNA accessibility is a differential genomic DNA accessibility compared to an expectation set of genomic DNA accessibility.

6. The method of claim 1, wherein the at least one ctDNA property is chosen from one or more of methylation, a transcriptome profile, nucleosome positioning, chromatin structure, 3D nucleus organization of a nucleus, a copy number variation, and expression levels of one or more genes.

7. (canceled)

8. (canceled)

9. (canceled)

10. (canceled)

11. (canceled)

12. (canceled)

13. The method of claim 6, wherein a gene of the one or more genes encodes a nuclease.

14. The method of claim 6, wherein a gene of the one or more genes encodes an apoptosis pathway member.

15. The method of claim 6, wherein a gene of the one or more genes encodes a necrosis pathway member.

16. The method of claim 1, wherein the at least one ctDNA property comprises base composition of nucleic acid fragment native ends.

17. The method of claim 1, wherein the at least one ctDNA property comprises genomic context of nucleic acid fragment native ends.

18. The method of claim 1, wherein the at least one ctDNA property comprises read depth coverage at one or more loci.

19. The method of claim 1, wherein the at least one ctDNA property comprises epigenetic protein modification.

20. The method of claim 19, wherein the epigenetic protein modification is histone methylation.

21. The method of claim 19, wherein the epigenetic protein modification is histone acetylation.

22. The method of claim 19, wherein the epigenetic protein modification is histone phosphorylation.

23. The method of claim 1, wherein the at least one ctDNA property comprises fragment length.

24. The method of claim 1, wherein the at least one ctDNA property comprises fragment overhang sequence.

25. The method of claim 1, wherein the at least one ctDNA property comprises fragment overhang length.

26. The method of claim 1, wherein the at least one ctDNA property comprises fragment overhang directionality.
